SEQUENCE LISTING (2 sequences)

SEQ ID NO: 1 - protein, 1172 aa, Arabidopsis thaliana
MALQNKEEEKKKVKKLQKSYFDVLGICCTSEVPIIENILKSLDGVKEYSVIVPSRTVIVVHDSLLISPFQIAKALNEARLEANVRVNGETSFKNKWPSPFAVVSGLLLLLSFLKFVYSPLRWLAVAAVAAGIYPILAKAFASIKRPRIDINILVIITVIATLAMQDFMEAAAVVFLFTISDWLETRASYKATSVMQSLMSLAPQKAIIAETGEEVEVDEVKVDTVVAVKAGETIPIDGIVVDGNCEVDEKTLTGEAFPVPKQRDSTVWAGTINLNGYICVKTTSLAGDCVVAKMAKLVEEAQSSKTKSQRLIDKCSQYYTPAIILVSACVAIVPVIMKVHNLKHWFHLALVVLVSGCPCGLILSTPVATFCALTKAATSGLLIKSADYLDTLSKIKIVAFDKTGTITRGEFIVIDFKSLSRDINLRSLLYWVSSVESKSSHPMAATIVDYAKSVSVEPRPEEVEDYQNFPGEGIYGKIDGNDIFIGNKKIASRAGCSTVPEIEVDTKGGKTVGYVYVGERLAGFFNLSDACRSGVSQAMAELKSLGIKTAMLTGDNQAAAMHAQEQLGNVLDVVHGDLLPEDKSRIIQEFKKEGPTAMVGDGVNDAPALATADIGISMGISGSALATQTGNIILMSNDIRRIPQAVKLARRARRKVVENVCLSIILKAGILALAFAGHPLIWAAVLVDVGTCLLVIFNSMLLLREKKKIGNKKCYRASTSKLNGRKLEGDDDYVVDLEAGLLTKSGNGQCKSSCCGDKKNQENVVMMKPSSKTSSDHSHPGCCGDKKEEKVKPLVKDGCCSEKTRKSEGDMVSLSSCKKSSHVKHDLKMKGGSGCCASKNEKGKEVVAKSCCEKPKQQVESVGDCKSGHCEKKKQAEDIVVPVQIIGHALTHVEIELQTKETCKTSCCDSKEKVKETGLLLSSENTPYLEKGVLIKDEGNCKSGSENMGTVKQSCHEKGCSDEKQTGEITLASEEETDDQDCSSGCCVNEGTVKQSFDEKKHSVLVEKEGLDMETGFCCDAKLVCCGNTEGEVKEQCRLEIKKEEHCKSGCCGEEIQTGEITLVSEEETESTNCSTGCCVDKEEVTQTCHEKPASLVVSGLEVKKDEHCESSHRAVKVETCCKVKIPEACASKCRDRAKRHSGKSCCRSYAKELCSHRHHHHHHHHHHHVSA

SEQ ID NO: 2 - protein, 1172 aa, Artificial
  AtHMA4 variant
MALQNKEEEKKKVKKLQKSYFDVLGICCTSEVPIIENILKSLDGVKEYSVIVPSRTVIVVHDSLLISPFQIAKALNEARLEANVRVNGETSFKNKWPSPFAVVSGLLLLLSFLKFVYSPLRWLAVAAVAAGIYPILAKAFASIKRPRIDINILVIITVIATLAMQDFMEAAAVVFLFTISDWLETRASYKATSVMQSLMSLAPQKAIIAETGEEVEVDEVKVDTVVAVKAGETIPIDGIVVDGNCEVDEKTLTGEAFPVPKQRDSTVWAGTINLNGYICVKTTSLAGDCVVAKMAKLVEEAQSSKTKSQRLIDKCSQYYTPAIILVSACVAIVPVIMKVHNLKHWFHLALVVLVSGXPCGLILSTPVATFCALTKAATSGLLIKSADYLDTLSKIKIVAFDKTGTITRGEFIVIDFKSLSRDINLRSLLYWVSSVESKSSHPMAATIVDYAKSVSVEPRPEEVEDYQNFPGEGIYGKIDGNDIFIGNKKIASRAGCSTVPEIEVDTKGGKTVGYVYVGERLAGFFNLSDACRSGVSQAMAELKSLGIKTAMLTGDNQAAAMHAQEQLGNVLDVVHGDLLPEDKSRIIQEFKKEGPTAMVGDGVNDAPALATADIGISMGISGSALATQTGNIILMSNDIRRIPQAVKLARRARRKVVENVCLSIILKAGILALAFAGHPLIWAAVLVDVGTCLLVIFNSMLLLREKKKIGNKKCYRASTSKLNGRKLEGDDDYVVDLEAGLLTKSGNGQCKSSCCGDKKNQENVVMMKPSSKTSSDHSHPGCCGDKKEEKVKPLVKDGCCSEKTRKSEGDMVSLSSCKKSSHVKHDLKMKGGSGCCASKNEKGKEVVAKSCCEKPKQQVESVGDCKSGHCEKKKQAEDIVVPVQIIGHALTHVEIELQTKETCKTSCCDSKEKVKETGLLLSSENTPYLEKGVLIKDEGNCKSGSENMGTVKQSCHEKGCSDEKQTGEITLASEEETDDQDCSSGCCVNEGTVKQSFDEKKHSVLVEKEGLDMETGFCCDAKLVCCGNTEGEVKEQCRLEIKKEEHCKSGCCGEEIQTGEITLVSEEETESTNCSTGCCVDKEEVTQTCHEKPASLVVSGLEVKKDEHCESSHRAVKVETCCKVKIPEACASKCRDRAKRHSGKSCCRSYAKELCSHRHHHHHHHHHHHVSA

(19) United States
(12) Patent Application Publication
(10) Pub. No.: US 2011/0023177 A1
(43) Pub. Date: Jan. 27, 2011

(54) Genetically Modified Plants Displaying Reduced Accumulation of Cadmium

(76) Inventors: Pierre Richaud, La Bastdonne (FR); Antoine Gravot, L'hermitagr (FR); Pascaline Auroy, Lauris (FR); Alain Vavasseur, Aix En Provence (FR)

Correspondence Address: MORGAN LEWIS & BOCKIUS LLP, 1111 PENNSYLVANIA AVENUE NW, WASHINGTON, DC, 20004, US

(21) Appl. No.: 12/746,725

(22) PCT Filed: Dec. 12, 2007
(86) PCT No.: PCT/IB07/04420

§ 371 Date: Oct. 7, 2010

(51) Int. Cl. C12N 15/82; C12N 9/14; C07H 21/00; C12N 5/10; A01H 5/00; A01H 5/12

(52) U.S. Cl. 800/278; 435/195; 536/23.2; 435/320.1; 435/419; 800/298; 800/317.3

(57) ABSTRACT

The present invention relates to methods and means such as polynucleotides, recombinant expression cassettes and recombinant vectors, for reducing the accumulation of cadmium in a plant, and a transgenic plant expressing a variant of a plant P_{1B}-type ATPase of the Zn²⁺/Co²⁺/Cd²⁺/Pb²⁺ subgroup, said transgenic plant displaying a reduced accumulation of cadmium in the aerial parts.

The present invention relates to means and methods for reducing the accumulation of cadmium in a plant, in particular tobacco.

Large cultivated areas present rising cadmium concentrations. This is due on the one hand to the natural high level of metal in certain soils, and on the other hand to the use of fertilizers (due to the presence of cadmium in the phosphate rock used for their manufacture), the spread of wastewater treatment station sludge, the field watering with urban wastewaters and the laying of aerosols. In the near future, the cadmium concentration in cultivated soils should continue to strongly increase due to the depletion of cadmiumless-phosphate mines used as fertilizers. As a consequence, the cadmium content found in plants, mainly those cultivated for foodstuffs, could be more and more frequently over the standards implemented by the different countries or recommended by the Food and Agriculture Organization of the United Nations. By way of example, this is the case for green salad, tomato, pepper, wheat and, particularly, tobacco. Regarding tobacco, this plant is known to accumulate high level of cadmium in leaves; the cadmium concentration in tobacco is reported in the literature as being 0.5-5 ppm.

Cadmium is a metallic element belonging to group IIB of the periodic table of elements. Due to its chemical toxicity, inhalation or ingestion of high level of cadmium can represent severe health risks for both human and animals (including damage to the respiratory system, kidney or liver, and cancer).

Therefore, there is a need for plants that can grow in a soil containing high cadmium concentration, and that are able to have a low content of this harmful metal. While different strategies may be followed to reduce cadmium concentration in plants, the production of transgenic plant may also contribute to obtain such plants.

A method for producing transgenic plants with enhanced resistance and decreased uptake of cadmium has been proposed by Lee et al. (2003, Plant Physiol. 133:589-96; and International Application No. WO 02/081707). The authors have obtained genetically modified Arabidopsis thaliana expressing Escherichia coli heavy metal ZntA protein. The transgenic plants displayed improved resistance to Pb and Cd, and the shoots thereof had decreased Pb and Cd content compared to the wild type.

ZntA is a P_{1B}-type ATPase that confers to E. coli resistance to toxic concentrations of divalent cations, such as Zn²⁺, Cd²⁺ and Pb²⁺, by active efflux of these metal ions outside the cytoplasm (Rensing et al., 1997, PNAS, 94:14326-14331; Rensing et al., 1998, J Biol Chem., 273:32614-32617; Dutta et al., 2007, Biochemistry, 46:3692-3703).

P_{1B}-ATPases are transporters that use the energy liberated by the exergonic ATP hydrolysis reaction to translocate across membranes soft metal cations, such as Zn²⁺, Cd²⁺, Pb²⁺, Co²⁺, Cu⁺ and Ag⁺ (Inesi, 1985, Annu Rev Physiol., 47:573-601; Axelsen and Palmgren, 2001, Plant Physiol., 126:696-706; and for review: Argüello et al., 2007, BioMetals, 20:233-248). They are sometimes referred to as HMAs (for Heavy Metal ATPases) or to as CPx-type ATPases. P_{1B}-ATPases are found in prokaryote and eukaryote organisms, including archaea, bacteria, yeasts, insects, plants and mammals. They all contain the DKTGT signature amino acid sequence. P_{1B}-ATPases are divided into two main distinct subgroups (clusters) based on the substrate cation selectivity and phylogenetic analyses (Rensing et al., 1999, J Bacteriol., 181:5891-5897). The Cu⁺ cluster P_{1B}-ATPases are involved in the transport of Cu⁺ and Ag⁺, whereas the Zn²⁺ cluster P_{1B}-ATPases transport Zn²⁺ and other heavy metals such as Co²⁺, Cd²⁺ and Pb²⁺ (Axelsen and Palmgren, 2001, above-cited). The different subgroups of P_{1B}-ATPases have distinct motifs of conserved amino acid in their transmembrane domains. By way of example, their sixth transmembrane domain contains a (C, S, T)P(C,H) motif depending on the cation selectively transported (Solioz and Vulpe, 1996, Trends Biochem Sci., 21:237-241; Rensing et al., 1998, above-cited; Williams et al., 2000, Biochim Biophys Acta., 1465:104-126; Dutta et al., 2007, above-cited).

In Arabidopsis thaliana, HMA2 and HMA4 proteins (AtHMA2 and AtHMA4 respectively) are P_{1B}-type ATPases that cluster with the Zn²⁺/Co²⁺/Cd²⁺/Pb²⁺ subgroup. AtHMA2 and AtHMA4 are localized in planta at the plasma membrane and are involved in the transport of these divalent cations to the aerial parts of the plant (Hussain et al., 2004, Plant Cell., 16:1327-39, Verret et al., 2004, FEBS Lett., 576:306-312). Zinc is an essential micronutrient required by the plants, while cobalt, cadmium and lead exhibit toxic effects. The amino acid sequence of AtHMA2 and AtHMA4 are available under accession numbers GI|12229675 and GI|12229637 respectively in the GenBank database; AtHMA4 amino acid sequence is reproduced herein as SEQ ID NO: 1. These proteins contain the conserved CPC motif (herein after also denoted C₁PC₂) in their sixth transmembrane domain and the DKTGT motif in the soluble loop between their sixth and seventh transmembrane domain (Argüello et al., 2007, above-cited). In AtHMA4, the first cysteine residue (C₁) is located at position 357 of the amino acid sequence (Mills et al., 2003, Plant J., 35:164-76). Mills et al. (2003, previously cited) showed that expression of AtHMA4 in Saccharomyces cerevisiae decreased the sensitivity of the yeast to Cd and conferred resistance to this metal ion. In a second article published in 2005 (FEBS Letters, 579:783-791), Mills et al. showed that expression in S. cerevisiae of an AtHMA4 mutant in which the first cysteine (C₁) of the CPC motif (³⁵⁷C) was substituted by a glycine (AtHMA4-C357G) did not confer Cd and Zn resistance to yeast, i.e., yeasts expressing AtHMA4-C357G were more sensitive to elevated levels of Cd and Zn than those expressing the wild type AtHMA4.

Transgenic A. thaliana overexpressing AtHMA4 have also been obtained (Verret et al., above-cited; and International Application No. WO 2005/090583). Contrary to the transgenic A. thaliana expressing ZntA (E. coli P_{1B}-type ATPase that clusters the Zn²⁺ subgroup) obtained by Lee et al. (see above), the transgenic A. thaliana obtained by Verret et al. displayed an increase in the Zn and Cd shoot content compared to the wild type.

It emerges from the foregoing that expression or overexpression in a transgenic plant of a P_{1B}-type ATPase that clusters the Zn²⁺ subgroup can lead to a different phenotype according to the prokaryote or plant origin of the P_{1B}-type ATPase.

Within the framework of research that has lead to the present invention, the Inventors have found, unexpectedly, that S. cerevisiae expressing an AtHMA4 variant in which the first cysteine (C₁) residue of the conserved C₁PC₂ motif in the sixth transmembrane domain was substituted by a serine (AtHMA4-C357S), exhibited diminished Cd transport capabilities while the Zn efflux was almost identical in comparison with the wild type yeast. Then, the Inventors have shown that a transgenic A. thaliana expressing the AtHMA4-C357S variant displayed a reduced uptake of cadmium compared to the wild type, while, advantageously, homeostasis of the physiological Zn²⁺ micronutrient was unmodified.

Accordingly, in a first aspect, the present invention provides a method for reducing the accumulation of cadmium in the aerial parts of a plant, characterized in that said method comprises expressing in said plant a variant of a plant P_{1B}-type ATPase of the Zn²⁺/Co²⁺/Cd²⁺/Pb²⁺ subgroup having the C₁PC₂ motif in the sixth transmembrane domain, and localised in planta at the plasma membrane, said variant having a mutation consisting of the substitution of the C₁ residue by any amino acid selected from the group consisting of serine, alanine, histidine and threonine, preferably serine.

The term “aerial parts” includes, but is not limited to, the shoots, leaves, stems, flowers, fruits and seeds, preferably the leaves and seeds.

The localization of said P_{1B}-type ATPase in planta at the plasma membrane can be determined by methods well-known from one of ordinary skill in the art. By way of example, one can cite the methods described in Hussain et al., 2004 and/or Verret et al., 2004 (both above-cited). Briefly, total plasma membranes from plants are fractioned by aqueous two-phase partitioning and the fractions are characterized by protein gel blot analysis probed with P_{1B}-type ATPase specific antibodies. To confirm the plasma membrane localization of said P_{1B}-type ATPase, the coding sequence thereof can be fused in frame with a marker enzyme, such as the green fluorescent protein (GFP) and expressed under control of the constitutive promoter, such as the CaMV 35S promoter, in transgenic plants or in protoplasts (transient expression). The subcellular localization of the P_{1B}-type ATPase-marker enzyme in cells can then be visualized by confocal microscopy.

According to a preferred embodiment of the invention, said P_{1B}-type ATPase is from a higher plant, such as A. thaliana, Nicotiana, Oryza and Populus, and more preferably from the same plant species than the plant in which said expression is desired.

In another preferred embodiment, said P_{1B}-type ATPase is selected from the group consisting of HMA4 and HMA2 from Arabidopsis thaliana, HMA4 from Thlaspi caerulescens (available under accession number GI|46361990 in the GenBank database), HMA4 from Arabidopsis halleri subsp. gemmifera (available under accession number GI|63056225 in the GenBank database), HMA2 and HMA3 from Oryza sativa (available under accession numbers GI|125598398 and GI|125557764 respectively in the GenBank database) and HMA from Vitis vinifera (available under accession number GI|157357491 in the GenBank database).

According to another particular embodiment of the invention, said P_{1B}-type ATPase has at least 50% preferably at least 54% and by order of increasing preference, at least 56%, 60%, 65%, 70%, 75%, 80%, 85%, 90%, 95%, 97%, 98% and 99% sequence identity, or at least 70%, preferably 73% and by order of increasing preference, at least 79%, 80%, 85%, 90%, 95%, 97%, 98% and 99% sequence similarity with the AtHMA4 protein of SEQ ID NO: 1, provided that it contains the conserved C₁PC₂ motif in the sixth transmembrane domain.

Unless otherwise specified, the protein sequence identity and similarity values provided herein are calculated using the BLASTP program under default parameters, on a comparison window including the whole sequence of the proteins. Similarity calculations are performed using the scoring matrix BLOSUM62.

Optionally, the substitutions defined above can be combined with one or more other mutation(s) aiming at improving the activity of these mutants.

According to another particular embodiment of the invention, said variant has the amino acid sequence SEQ ID NO: 2. This amino acid sequence corresponds to the wild type AtHMA4 protein (SEQ ID NO: 1) in which the first cysteine (C₁) residue of the conserved C₁PC₂ motif in the sixth transmembrane domain of the protein is substituted by an amino acid as described above (i.e., serine, alanine, histidine or threonine).

According to another embodiment of the invention, the method for reducing the accumulation of cadmium in a plant further comprises the inhibition in said plant of at least one, preferably all, endogenous (wild type) P_{1B}-type ATPase(s) of the Zn²⁺/Co²⁺/Cd²⁺/Pb²⁺ subgroup having the C₁PC₂ motif in the sixth transmembrane domain and localized in planta at the plasma membrane.

The method according to this embodiment only concerns plants expressing said endogenous P_{1B}-type ATPase(s). Indeed, plants may express one or several endogenous P_{1B}-type ATPases as defined above. By way of example, A. thaliana expresses the P_{1B}-type ATPase HMA2 and HMA4. Therefore, when the method according to this embodiment is applied to A. thaliana, then the method comprises the inhibition of HMA2 and/or HMA4.

The inhibition of an endogenous P_{1B}-type ATPase can be obtained either by abolishing, blocking or decreasing its function, or advantageously, by preventing or down-regulating the expression of its gene.

By way of example, inhibition of said endogenous P_{1B}-type ATPases can be obtained by mutagenesis of the corresponding genes or of their promoters, and selection of the mutants having partially or totally lost the P_{1B}-type ATPase activity. For instance, a mutation within the coding sequence can induce, depending on the nature of the mutation, the expression of an inactive protein; in the same way, a mutation within the promoter sequence can induce a lack of expression of said endogenous P_{1B}-type ATPases, or decrease thereof.

Mutagenesis can be performed for instance by targeted deletion of the endogenous P_{1B}-type ATPase coding sequences or promoters, or of a portion thereof, or by targeted insertion of an exogenous sequence within said coding sequences or said promoter(s). It can also be performed by random chemical or physical mutagenesis, followed by screening of the mutants within the gene encoding said endogenous P_{1B}-type ATPases. Methods for high throughput mutagenesis and screening are available in the art. One can cite, for example, the method described in Hussain et al., 2004 (above-cited) in which a T-DNA was inserted in the hma2 and hma4 alleles from A. thaliana and the mutants were grown on a medium containing high concentration of Zn (these mutants having Zn deficiency).

Advantageously, the inhibition of said endogenous P_{1B}-type ATPases is obtained by silencing of the corresponding genes. Methods for gene silencing in plants are known in themselves in the art. For instance, one can mention by antisense inhibition or co-suppression. It is also possible to use ribozymes targeting the mRNA of said endogenous P_{1B}-type ATPases.

Preferred methods are those wherein post transcriptional gene silencing is induced by means of RNA interference (RNAi) targeting the genes encoding said endogenous P_{1B}-type ATPases to be silenced. Various methods and DNA constructs for delivery of RNAi are available in the art (for review: Watson et al., 2005, FEBS Letters, 579:5982-5987).

The instant invention also provides a variant of a P_{1B}-type ATPase protein, as defined above.

The instant invention also provides means for carrying out said expression.

Thus, the present invention provides polynucleotides encoding a variant of a P_{1B}-type ATPase as defined above. The polynucleotides of the invention may be obtained by the well-known methods of recombinant DNA technology and/or of chemical DNA synthesis. These methods also allow to introduce the desired substitution in a naturally occurring nucleotide sequence encoding a P_{1B}-type ATPase as defined above.

The present invention also provides recombinant expression cassettes comprising a polynucleotide encoding a variant of a P_{1B}-type ATPase as defined above under control of a transcriptional promoter allowing the regulation of said polynucleotide in a host cell.

According to a preferred embodiment, said transcriptional promoter is any promoter functional in a plant cell, i.e., capable of directing transcription of a polynucleotide encoding a variant of a P_{1B}-type ATPase as defined above, in a plant cell. The choice of the more appropriate promoter may depend in particular on the organ(s) or tissue(s) targeted for expression, and on the type of expression (i.e., constitutive or inducible) that one wishes to obtain. A large choice of promoters suitable for expression of genes in plants, and in particular in tobacco, is available in the art. They can be obtained for instance from plants (e.g., A. thaliana and N. tabacum), plant viruses, or bacteria such as Agrobacterium. They include constitutive promoters, i.e. promoters which are active in most tissues and cells and under most environmental conditions, tissue or cell specific promoters which are active only or mainly in certain tissues (e.g., leaves) or certain cell types, and inducible promoters that are activated by physical or chemical stimuli. They also include the promoter of said endogenous P_{1B}-type ATPase from the same plant species than the plant in which said expression is desired. The sequence of the promoters can also be repeated two or more times (e.g., duplicated in tandem).

Non-limitative examples of constitutive promoters that are commonly used are the well-known cauliflower mosaic virus (CaMV) 35S promoter and the nopaline synthase (Nos) promoter, the Cassava vein Mosaic Virus (CsVMV) promoter (Verdaguer et al., 1996, Plant Mol. Biol., 31:1129-39), the rice actin (1 or 2) promoter followed by the rice actin intron (RAP-RAI) contained in the plasmid pAct1-F4 used for transgenic monocotyledon plants (McElroy et al., 1991, Mol. Gen. Genet., 231(1):150-160).

The expression cassettes generally also include a transcriptional terminator, such as the 35S transcriptional terminator or Nos terminator (Depicker et al., 1982, J. Mol. Appl. Genet., 1:561-73). They may also include other regulatory sequences, such as transcription enhancer sequences.

The recombinant expression cassettes of the invention can be inserted in an appropriate vector allowing genetic transformation of the genome of a host cell.

Thus, the present invention also relates to recombinant vectors containing an expression cassette as defined above, the promoter of said expression cassette being preferably a promoter functional in a plant cell.

The present invention also provides host cells containing a recombinant expression cassette or a recombinant vector as defined above.

The host cells of the present invention are prokaryotic cells or eukaryotic cells, preferably plant cells, and more preferably tobacco cells.

In another aspect, the present invention provides a method for producing a transgenic plant having a reduced cadmium accumulation. Said method comprises the following steps:

a) providing a plant cell containing a recombinant expression cassette or a recombinant vector as defined above, and optionally in which the endogenous P_{1B}-type ATPase as defined above is inhibited, and

b) regenerating from the plant cell obtained in step a) a transgenic plant expressing a variant of a P_{1B}-type ATPase as defined above.

Owing to the use of a variant of a P_{1B}-type ATPase of plant origin, the method of the invention has the advantage that the transgenic plants produced contain a minimal content of xenogenetic elements or foreign sequences, which makes the expression of said variant more stable and more efficient.

The invention also comprises plants genetically transformed by a recombinant expression cassette of the invention, and expressing a variant of a P_{1B}-type ATPase as defined above. Preferably, said transgenic plants are obtainable by a method of the invention. In said transgenic plants, a recombinant expression cassette of the invention is comprised in one or several transgene(s) integrated (i.e., stably integrated) in the plant genome, so that it is passed onto successive plant generations. Thus the transgenic plants of the invention include not only the plants resulting from the initial transgenesis, but also their descendants, as far as they contain a recombinant expression cassette of the invention.

Advantageously, a transgenic plant of the invention expressing a variant of a P_{1B}-type ATPase as defined above displays a reduced uptake of cadmium, particularly in the aerial parts, when compared with a plant of the same species devoid of said transgene.

Accordingly, the invention provides a transgenic plant or an isolated organ (such as seeds, leaves, flowers, roots, stems, ears, preferably leaves) or tissue thereof comprising, stably integrated in its genome, a recombinant expression cassette comprising a polynucleotide encoding a variant of a P_{1B}-type ATPase as defined above.

The present invention applies to monocot- or dicotyledon plants of agronomical interest, such as wheat, barley, corn, rape, rice, chard, spinach, lettuce, tomato, tobacco, preferably tobacco.

Tobacco leaves naturally accumulate and concentrate relatively high levels of cadmium, which is volatilized during burning, and contributes significantly to a smoker's exposure to cadmium. Advantageously, a tobacco leaf from a transgenic plant of the invention has lower cadmium content when compared to a wild type tobacco or a tobacco devoid of said transgene(s) defined above.

Accordingly, in another aspect, the present invention relates to the use a tobacco leaf from a transgenic plant of the invention for the manufacture of tobacco products including smoking products, such as cigarettes, cigars and shags, as well as smokeless products, such as snuffing, chewing, or sucking tobacco, and the like. Said products are also encompassed by the present invention.

Foregoing and other objects and advantages of the invention will become more apparent from the following detailed description and accompanying drawings. It is to be understood however that this foregoing detailed description is exemplary only and is not restrictive of the invention.

### DESCRIPTION OF THE DRAWINGS

FIG. 1: a) Growth of pYES-only, AtHMA4- and AtHMA4SPC-yeast transformants in liquid media followed by measuring optical density (OD) at 600 nm. Yeast cells were grown at 30° C. for 48 h in the presence of Cd 80 μM. b-d) Liquid cultures were carried out in the same conditions as in a). The cells were collected, washed one time with 10 mM EDTA then washed two times with demineralised water to remove the cadmium adsorbed on the yeast cell walls. The pellets were dried, weighted, mineralised with HNO₃ then the metal content was determined by ICP-AES; in b) and d) the yeast cultures were carried out in the presence of Cd 80 μM; in c) the yeast cultures were carried out in a controlled nutrient solution (1.5 μM Zn).

FIG. 2 shows the in vitro root growth measurement of A. thaliana Wassilewskija (Ws) and transgenic A. thaliana overexpressing AtHMA4 (CPC) or AtHMA4SPC(SPC). Plantlets were grown vertically on bactoagar nutrient solution in the absence (control) or in the presence of the following metals, 20 μM Cd, 50 μM Zn or 200 μM Zn. The root length measurements were carried out 14 days after germination. The Figure indicates the mean values of 100-150 measures +/−SD.

### EXAMPLE

### Characterization of Transgenic Yeast and Arabidopsis Thaliana Expressing a Variant Form of AtHMA4

1) Material and Method:

The HMA4 cDNA from Arabidopsis thaliana (accession number AF412-407 in the GENBANK database, Marsh 10, 2005 version), cloned in the inducible yeast vector pYES-GFP (Gravot et al., 2004, FEBS Lett., 561:22-28.), was modified by site-directed mutagenesis. The amino acid residue cysteine at position 357 in the sequence was substituted by a serine (clone pVF472) to obtain the amino acid sequence SEQ ID NO: 2 in which is Xaa is the amino acid serine (named AtHMA4SPC).

This construction was transformed in the yeast mutant strains hypersensitive to Cd and Pb (ycf1) (Li et al., 1996, J Biol Chem., 271:6509-6517) and to Zn (zrc1) (MacDiarmid et al., 2003, J Biol Chem., 278:15065-15072) respectively (clones pVF4121 and pVF4136, respectively) for heterologous experiments.

The insert of pVF472 was adapted by PCR for cloning in a plant vector presenting the strong ectopic promoter CaMV35S (clones pAP4152 and pAP4154). The vector pAP4154 was introduced in Agrobacterium tumefaciens strain AglI.

A. thaliana plants (ecotype Wassilewskija) were transfected by floral dip (Clough and Bent, 1998, Plant J., 16:735-743). The transformants were selected on hygromycin. Several independent lines of homozygous plants presenting one insertion of the construction were phenotypically characterized. Plants of wild type (Ws) and overexpressing the variant form of AtHMA4 (AtHMA4SPC) were grown on solid control or in a medium containing various cations transported by AtHMA4. Seeds were germinated in a controlled-environment (8 h photoperiod at 300 μmol m⁻² s⁻¹, 21° C. and 70% relative humidity). The root length was measured 14 days after germination. T3 plants (homozygous plants carrying a unique T-DNA) were used for phenotypic characterization.

A. thaliana overexpressing AtHMA4 were obtained as described in Verret et al. 2004, FEBS Lett., 576:306-312.

2) Results:

2-1) Characterization of the Transfected Yeasts:

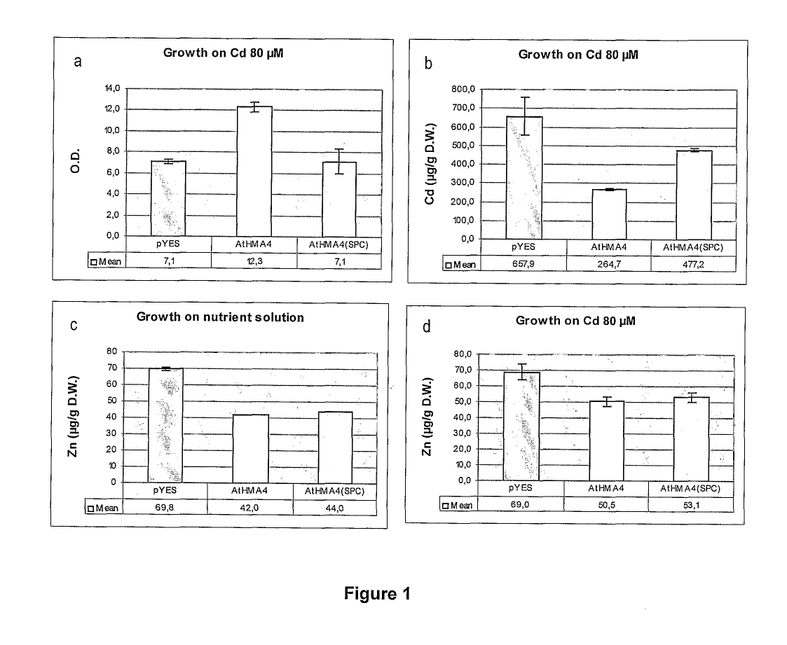

2-1-1) The wild type yeast Saccharomyces cerevisiae was transformed with the empty vector (pYES), the cDNA encoding the wild type HMA4 from A. thaliana (AtHMA4, SEQ ID NO: 1), or the cDNA encoding a variant of AtHMA4 where the cystein at position 357 has been substituted with a serine (AtHMA4SPC, SEQ ID NO: 2). Liquid cultures in the presence of Cd 80 μM were carried out at 30° C. and the O.D. was followed at 600 nm during 48 hours. In agreement with the results obtained by Verret et al. (2005, FEBS Lett., 579:1515-1522), a better growth of the yeast transformed with the wild type AtHMA4 was observed in the presence of Cd 80 μM. AtHMA4 induced an increased tolerance toward cadmium. When transformed with the variant form (AtHMA4SPC), the yeast did not present this increased tolerance and the growth was identical to the control strain. Results are shown in FIG. 1a.

2-1-2) The metal contents in yeast were determined by ICP-AES as described by Gravot et al. (2004, above-cited). Results are shown in FIG. 1b-d.

As previously described by Verret et al., 2005 (above-cited), Cd concentration in yeast expressing AtHMA4 was found greatly lower (60%) than the Cd concentration in the control strain (FIG. 1b). AtHMA4 was expressed at the plasma membrane of yeasts and had a role in an ATP-dependent Cd/Zn efflux.

The Cd concentration in yeast expressing AtHMA4SPC was halfway between both other strains (27.5% lower than the control). A lower capability to transport and efflux Cd could explain this observation.

The Zn concentration was measured in two growth conditions: in nutrient solution containing 1.5 μM Zn, and in the presence of Cd 80 μM. In both cases, the Zn concentration was identical in AtHMA4- and AtHMA4SPC-transformed yeasts and lower than in control yeasts (FIGS. 1c and 1d).

These experiments show that the Zn transport function of AtHMA4 was not modified by the C357S substitution, even in the presence of a toxic concentration of Cd, whereas the Cd transport capability was diminished.

2-2) Characterization of the Transfected A. thaliana:

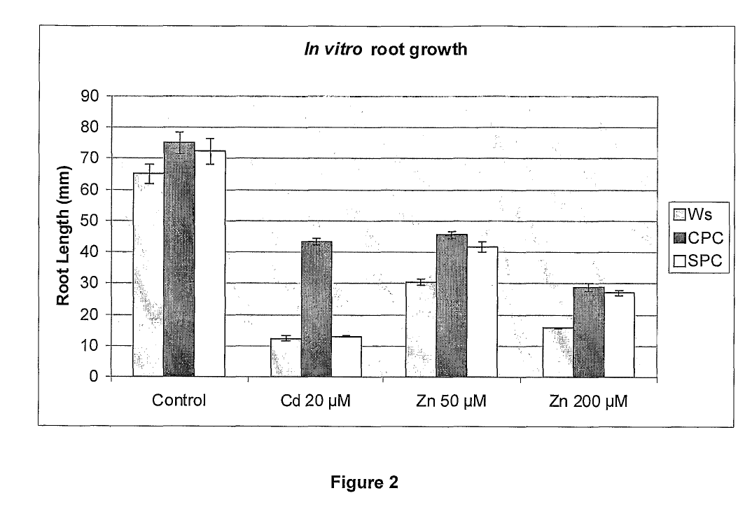

The plant tolerance toward the various metal transported by AtHMA4 or AtHMA4SPC was determined by the measure of the root length on bactoagar solid medium. The root length was the same for both genotypes in control condition. Results are shown in FIG. 2.

In the presence of different concentrations of Zn, Co and Pb, the root length was greater for seedlings overexpressing AtHMA4SPC than for the wild type ones. These results were in agreement with the results obtained in yeast, and with the observations made by Verret et al. (above-cited) in plants overexpressing the native form of AtHMA4. Plants overexpressing the variant form of AtHMA4 (AtHMA4SPC) present, as the ones overexpressing the native form of AtHMA4, an increased tolerance toward these three metals. This is due to more important metal transport and translocation to their shoot capabilities.

In the presence of Cd 20 μM, the root length was the same for both lines (wild type and variant). The increased tolerance toward cadmium also observed for the plants overexpressing AtHMA4 was lost. This result is in agreement with those obtained with yeasts.

To conclude, a variant of a P_{1B}-type ATPase of the Zn²⁺/Co²⁺/Cd²⁺/Pb²⁺ subgroup of eukaryotic origin having the C₁PC₂ motif in the sixth transmembrane domain (e.g., AtHMA4), said variant having a mutation consisting of the substitution of the C₁ residue by a serine, presents a modified metal transport specificity probably due to a decreased affinity toward cadmium. However, this property is unchanged toward the other three transported metals Co, Pb and especially the physiological Zn.

1. A method for reducing the accumulation of cadmium in the aerial parts of a plant, wherein said method comprises expressing in said plant a variant of a plant P_{1B}-type ATPase of the Zn²⁺/Co²⁺/Cd²⁺/Pb²⁺ subgroup having the C₁PC₂ motif in the sixth transmembrane domain and localised in planta at the plasma membrane, said variant having a mutation consisting of the substitution of the C₁ residue by any amino acid selected from the group consisting of serine, alanine, histidine and threonine.

2. The method according to claim 1, wherein said P_{1B}-type ATPase is from a higher plant.

3. The method according to claim 2, wherein said P_{1B}-type ATPase is from the same plant species than the plant for which said expression is desired.

4. The method according to claim 1, wherein said P_{1B}-type ATPase is selected from the group consisting of HMA4 and HMA2 from Arabidopsis thaliana, HMA4 from Thlaspi caerulescens, HMA4 from Arabidopsis halleri subsp. gemmifera, HMA2 and HMA3 from Oryza sativa and HMA from Vitis vinifera.

5. The method according to claim 4, wherein the variant of a P_{1B}-type ATPase has the amino acid sequence SEQ ID NO: 2.

6. The method according to claim 1, wherein it further comprises the inhibition of at least one endogenous P_{1B}-type ATPase of the Zn²⁺/Co²⁺/Cd²⁺/Pb²⁺ subgroup having the C₁PC₂ motif in the sixth transmembrane domain and localised in planta at the plasma membrane.

7. A variant of a plant P_{1B}-type ATPase of the Zn²⁺/Co²⁺/Cd²⁺/Pb²⁺ subgroup having the C₁PC₂ motif in the sixth transmembrane domain and localised in planta at the plasma membrane, said variant having a mutation consisting of the substitution of the C₁ residue by any amino acid selected from the group consisting of serine, alanine, histidine and threonine.

8. A polynucleotide encoding a variant of a P_{1B}-type ATPase of claim 7.

9. A recombinant expression cassette, comprising a polynucleotide as defined in claim 8, under control of a transcriptional promoter.

10. A recombinant vector, comprising a recombinant expression cassette as defined in claim 9, the promoter of said expression cassette being a promoter functional in a plant cell.

11. A host cell, comprising a recombinant expression cassette of claim 9.

12. A host cell according to claim 11, wherein it is a plant cell.

13. A method for producing a transgenic plant having a reduced cadmium accumulation, comprising the following steps:
a) providing a plant cell of claim 12, in which the endogenous P_{1B}-type ATPase is inhibited, and
b) regenerating from the plant cell obtained in step a) a transgenic plant expressing a variant of a P_{1B}-type ATPase.

14. A transgenic plant produced by the method of claim 13.

15. A transgenic plant or an isolated organ thereof or tissue thereof, comprising, stably integrated in its genome, a recombinant expression cassette as defined in claim 9.

16. The transgenic plant according to claim 14, wherein it is a transgenic tobacco plant.

17. An isolated organ according to claim 15, wherein it is a tobacco leaf.

18. (canceled)

19. A host cell comprising a recombinant vector of claim 10.
